(19) United States
(12) Patent Application Publication
(10) Pub. No.: US 2016/0151037 A1
(43) Pub. Date: Jun. 2, 2016

(54) STETHOSCOPE DEVICE

(71) Applicant: DailyCare Biomedical Inc, Taoyuan County (TW)

(72) Inventors: Kang-Ping LIN, Taoyuan County (TW); Bor-Iuan JAN, Taoyuan County (TW); Hao-Yu CHAN, Taoyuan County (TW); Keng-Hung LIN, Taoyuan County (TW); Yu-Hong YAN, Yunlin County (TW); Sheng-Kai YANG, Yilan County (TW)

(21) Appl. No.: 14/953,045

(22) Filed: Nov. 27, 2015

(30) Foreign Application Priority Data
Nov. 28, 2014 (TW) 103141364

(51) Int. Cl. A61B 7/04; A61B 5/04; A61B 5/00; A61B 5/0456

(52) U.S. Cl. CPC A61B 7/04; A61B 5/0456; A61B 5/04012; A61B 5/7405; A61B 5/742

(57) ABSTRACT

A stethoscope device is disclosed, which comprises a heart sound sensor comprising a sensing portion to detect a heart sound signal having a periodic first heart sound and a periodic second heart sound; an electrocardiogram sensor comprising two electrodes to detect an electrocardiogram signal having a periodic R wave; a microprocessor for extracting amplitudes of the R wave from the electrocardiogram signal and analyzing appearing positions of the R wave to provide appearing positions of a periodic notification pulse wave to a predetermined reference signal and combine the reference signal with the heart sound signal to form a sound signal; and an output unit to output the sound signal; wherein each pulse of the notification pulse wave of the sound signal provides a notification sound.

### CROSS REFERENCE TO RELATED APPLICATION

This application claims the benefits of the Taiwan Patent Application Serial Number 103141364, filed on Nov. 28, 2014, the subject matter of which is incorporated herein by reference.

### BACKGROUND OF THE INVENTION

1. Field of the Invention

The present invention relates to a stethoscope device, and more particularly, to a stethoscope device with a notification function.

2. Description of Related Art

The stethoscope is a widely used device to determine whether the condition of the heart is normal. Heart sounds are listened by the stethoscope by placing the sensing portion of the stethoscope against the heart. In general, heart sounds can be divided into first heart sound (S1), second heart sound (S2), third heart sound (S3), and fourth heart sound (S4). The first heart sound and the second heart sound can be listened to more easily. The first heart sound is produced by the closing of the atrioventricular valves. The second heart sound is produced by the closing of the semilunar valves. By listening to the intensities, frequencies, and regularities of the first heart sound and the second heart sound, the condition of the heart can be determined.

Since the frequencies of heartbeats can be fast and slow, it is rather difficult for doctors or beginners to determine when a heart sound has begun when a heart sound is heard. This causes the determination of the condition of the heart to be inaccurate. Thus, there is a need to provide an improved stethoscope device with a notification function to notify when a heart sound will begin.

### SUMMARY OF THE INVENTION

An object of the present invention is to provide a stethoscope device with a notification function, comprising: a heart sound sensor comprising a sensing portion to detect a heart sound signal having a periodic first heart sound and a periodic second heart sound; an electrocardiogram (ECG) sensor comprising two electrodes to detect an electrocardiogram signal having a periodic R wave; a microprocessor with a predetermined reference signal having a periodic notification pulse wave for extracting amplitudes of the R wave from the electrocardiogram signal and analyzing appearing positions of the R wave to provide appearing positions of the notification pulse wave to the reference signal and combine the reference signal with the heart sound signal to form a sound signal; and an output unit to output the sound signal; wherein each pulse of the notification pulse wave of the sound signal provides a notification sound. Since the notification sound will notify when a heart sound will begin; hence, result of auscultation will no longer be inaccurate.

Other objects, advantages, and novel features of the invention will become more apparent from the following detailed description when taken in conjunction with the accompanying drawings.

### BRIEF DESCRIPTION OF THE DRAWINGS

FIG. 1 is a schematic diagram of a framework of a stethoscope device with a notification function of the present invention;

FIG. 2 is a schematic diagram of an outer appearance of a stethoscope device with a notification function of the present invention;

FIG. 3 is a schematic diagram of a heart sound signal of the present invention;

FIG. 4 is a schematic diagram of an electrocardiogram signal of the present invention;

FIG. 5 is a flowchart of a procedure performed by the microprocessor of the present invention;

FIG. 6 is a schematic diagram of a reference signal of the present invention; and

FIG. 7 is a schematic diagram of a combined sound signal of the present invention.

### DETAILED DESCRIPTION OF THE PREFERRED EMBODIMENT

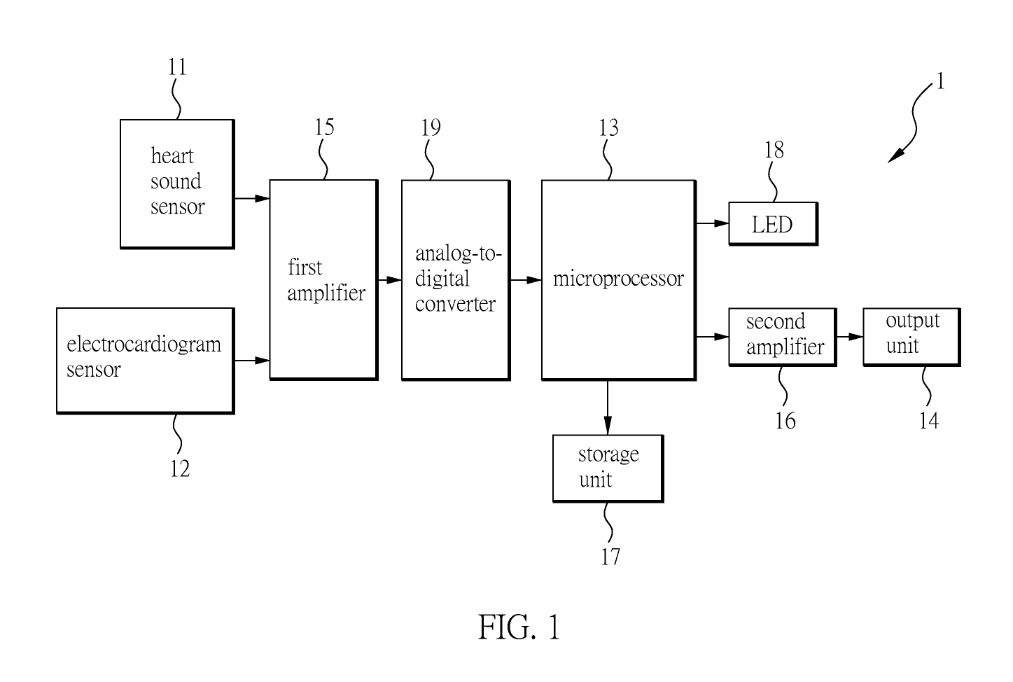

Please refer to FIG. 1. FIG. 1 is a schematic diagram of a framework of a stethoscope device with a notification function of the present invention. As shown in FIG. 1, the stethoscope device 1 comprises a heart sound sensor 11, an electrocardiogram sensor 12, a microprocessor 13, and an output unit 14. The heart sound sensor 11 and the electrocardiogram sensor 12 are coupled to the microprocessor 13 individually. This allows the signals detected by the heart sound sensor 11 and the electrocardiogram sensor 12 to be transmitted to the microprocessor 13. The microprocessor 13 is coupled to the output unit 14. This allows the signals processed by the microprocessor 13 to be transmitted to the output unit 14. A first amplifier 15 and an analog-to-digital converter 19 can be disposed among the heart sound sensor 11, the electrocardiogram sensor 12, and the microprocessor 13. This allows the signals detected by the heart sound sensor 11 and the electrocardiogram sensor 12 to be amplified by the first amplifier 15. The detected signals can be converted from analog signals to digital signals by the analog-to-digital converter 19. A second amplifier 16 can be disposed between the microprocessor 13 and the output unit 14. This allows the signals processed by the microprocessor 13 to be amplified by the second amplifier 16.

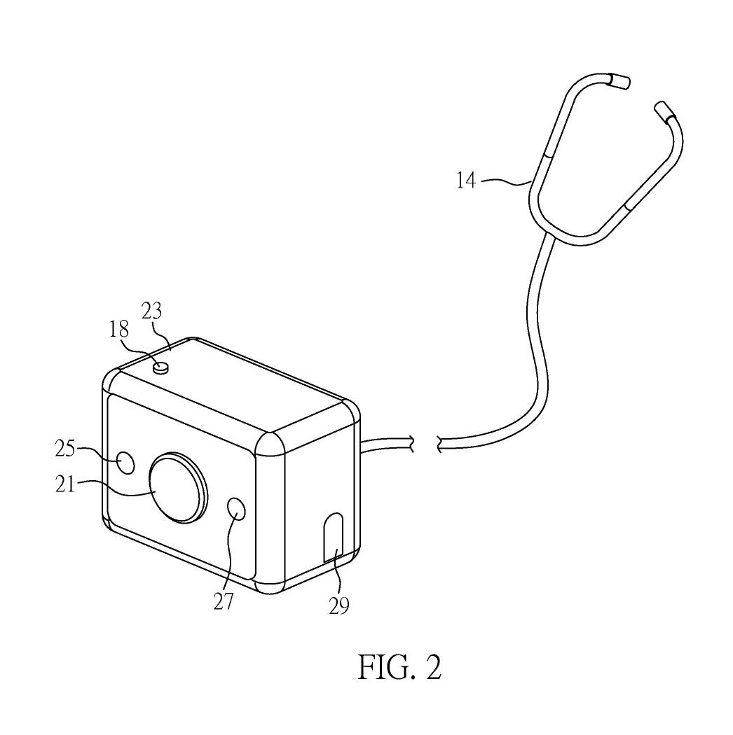

Please refer to FIG. 2. FIG. 2 is a schematic diagram of an outer appearance of a stethoscope device with a notification function of the present invention. As shown in FIG. 2, the heart sound sensor 11, the electrocardiogram sensor 12, and the microprocessor 13 are all disposed inside a housing 23. The output unit 14 is the headphone of the stethoscope device. The heart sound sensor 11 comprises a sensing portion 21 exposed from the housing 23 to detect, receive, and measure a heart sound signal. More specifically, the heart sound signal is obtained by placing the sensing portion 21 against any body parts such as the heart, chest, etc. Preferably, the sensing portion 21 is the sensing portion of a stethoscope. The electrocardiogram sensor 12 comprises two electrodes 25, 27 exposed from the housing 23 to detect, receive, and measure an electrocardiogram signal. More specifically, the electrocardiogram signal is obtained by placing the two electrodes 25, 27 against any body parts such as the chest, arms, legs, etc. Preferably, the two electrodes 25, 27 are disposed at two sides of the sensing portion 21 and at the edges of the housing 23.

Please refer to FIG. 3. FIG. 3 is a schematic diagram of a heart sound signal of the present invention. As shown in FIG. 3, the heart sound signal is an indirect continuous periodic signal having a periodic first heart sound, a periodic second heart sound, a periodic third heart sound, and a periodic fourth heart sound. Based on the intensities, frequencies, and regularities of the first heart sound and the second heart sound, the condition of the heart can be determined.

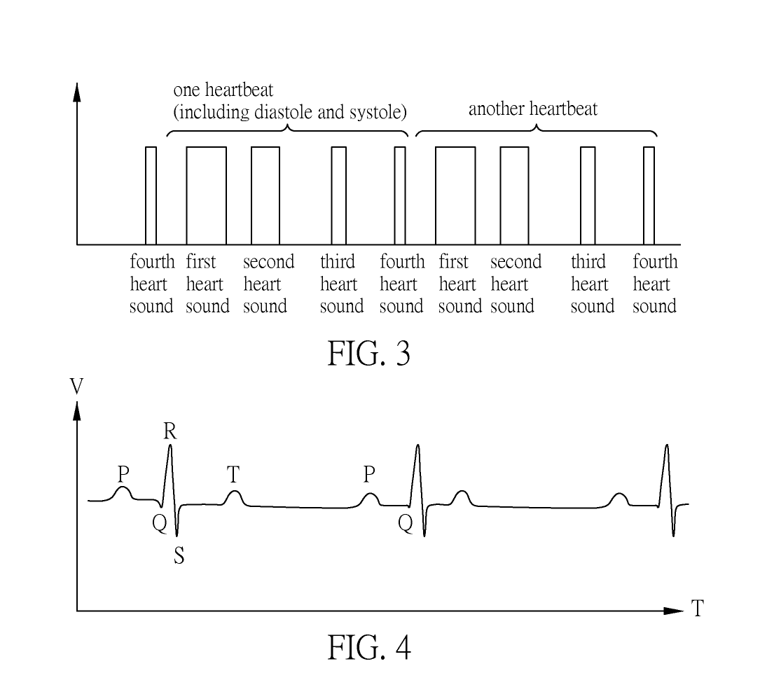

Please refer to FIG. 4. FIG. 4 is a schematic diagram of an electrocardiogram signal of the present invention. As shown in FIG. 4, the electrocardiogram signal has a periodic R wave. More specifically, the electrocardiogram signal has a periodic P wave, a periodic QRS complex, and a periodic T wave. The electrocardiogram signal will assist in the determination of the condition of the heart.

Please refer back to FIGS. 1 and 2. The microprocessor 13 is preferably a hardware capable of executing a software. The microprocessor 13 comprises an input terminal to input the heart sound signal detected by the heart sound sensor 11 and the electrocardiogram signal detected by the electrocardiogram sensor 12. The microprocessor 13 performs a procedure including analyzing and processing the heart sound signal and the electrocardiogram signal as well as combining the heart sound signal with a reference signal having a notification sound signal processed beforehand to a single sound signal. The microprocessor 13 also comprises an output terminal to output the sound signal to the output unit 14.

The output unit 14 can be any devices used for displaying signals. Preferably, the output unit 14 is a sound output device, such as a headphone, but it is certainly not limited thereto.

The stethoscope device 1 can further comprise several additional components, such as a storage unit 17. Preferably, the storage unit 17 is configured to couple to the microprocessor 13 to store signals such as the heart sound signal, the electrocardiogram signal, and the sound signal. The storage unit 17 can be any units with a storage function. Preferably, the storage unit 17 is a memory card such as a SD card, but it is certainly not limited thereto. The stethoscope device 1 can further comprise a slot 29 disposed on the housing 23. By inserting the storage unit 17 into the slot 29, the storage unit 17 will couple to the microprocessor 13.

The stethoscope device 1 can further comprise a LED 18. The LED 18 is connected to the microprocessor 13 and disposed on the housing 23. The LED 18 will emit light or flash light in correspondence to the sound signal to notify a heart sound will begin.

All of the above-mentioned signals can be amplified by the first amplifier 15 and the second amplifier 16 to ensure the processing and output of the signals to be more accurate and clearer.

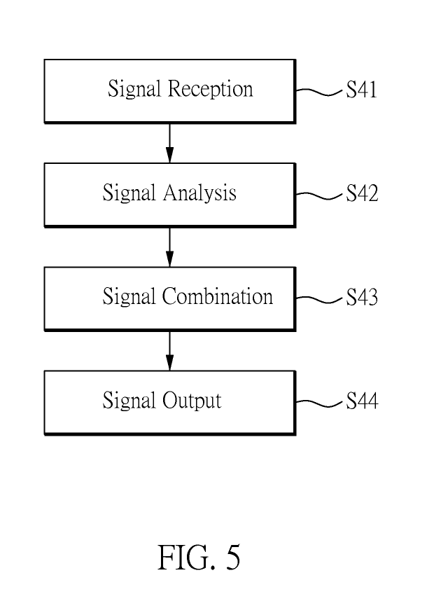

Please refer to FIG. 5. FIG. 5 is a flowchart of a procedure performed by the microprocessor of the present invention. More specifically, the procedure is a signal processing procedure comprising steps of S41, S42, S43, and S44. In step S41, the microprocessor 13 receives the heart sound signal and the electrocardiogram signal. These signals can be amplified by the first amplifier 15 before being received by the microprocessor 13 to assure more accurate signal processing. If these signals are analog signals, then the analog-to-digital converter 19 will convert these analog signals to digital signals before being received by the microprocessor 13.

In step S42, the microprocessor 13 analyzes the heart sound signal and the electrocardiogram signal individually. More specifically, the microprocessor 13 analyzes the amplitudes of the R wave from the electrocardiogram signal and records the time points of the appearing positions of the amplitudes of the R wave. The extraction of information regarding the R wave is a technique known in the art. Since the extraction of information regarding the R wave is not a major subject of the present invention, the extraction of information regarding the R wave will not be described here. Any techniques for extracting information regarding the R wave can be used in the present invention. The results of the signal analyses performed by the microprocessor 13 will be stored in a storage area of the microprocessor 13 or in the storage unit 17.

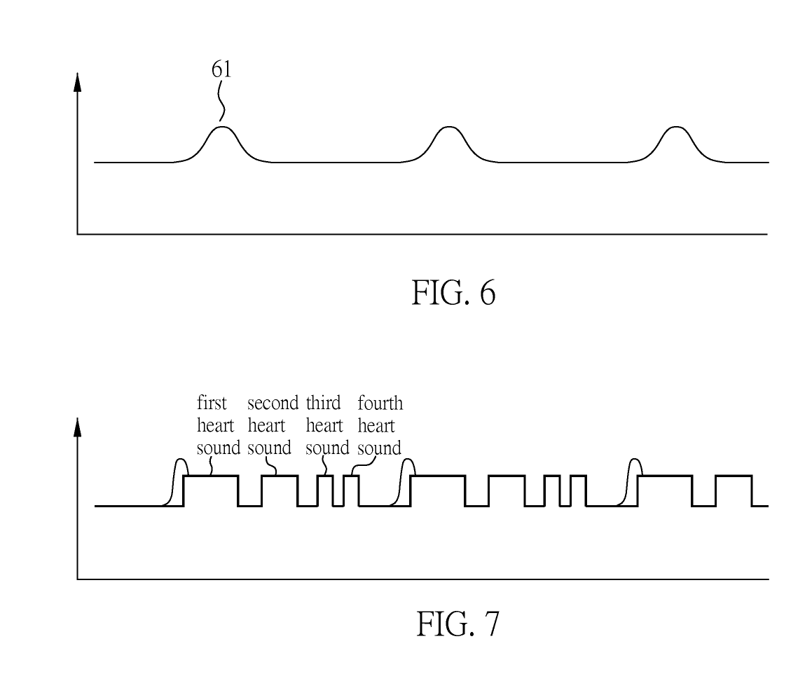

In step S43, the microprocessor 13 processes a predetermined reference signal according to the time points of the appearing positions of the R wave. FIG. 6 is a schematic diagram of a reference signal of the present invention. As shown in FIG. 6, the processed reference signal has a periodic notification pulse wave 61. Each appearing position of the notification pulse wave 61 is determined by each appearing position of the R wave. The microprocessor 13 combines the processed reference signal having the periodic notification pulse wave 61 with the heart sound signal to form a new signal. Preferably, the new signal is a sound signal and each pulse of the notification pulse wave of the sound signal provides a notification sound. The notification pulse wave 61 of the reference signal delays for a predetermined time compared to the corresponding R wave. The predetermined time is a time after the R wave has appeared and before the T wave appears. Preferably, the predetermined time is in the range of 0.06 seconds to 0.07 seconds. More preferably, the predetermined time is 0.065 seconds. However, the predetermined time is certainly not limited thereto. The time points at which the R wave appears often associate with the first heart sound (the heart sound usually appears slightly later than the R wave). Therefore, the notification sound precedes the first heart sound. The time points at which the notification sound begins are shown in FIG. 7, which is a schematic diagram of a combined sound signal of the present invention.

In step S44, the microprocessor 13 outputs the sound signal to the output unit 14. The sound signal can be amplified by the second amplifier 16 to assure that the sound signal outputted by the output device 14 will be clearer.

The microprocessor 13 can link the notification pulse wave 61 with the LED 18 so that the LED 18 emits light or flashes light when the notification pulse wave 61 appears. Thereby, the flashing of the LED 18 can notify a heart sound will begin together the notification sound.

Accordingly, the stethoscope device provided by the present invention has a notification function to notify a heart sound will begin. Specifically, the output unit of the stethoscope device will output a sound signal with a notification sound preceding the first heart sound. The notification sound will draw attention to hearing a heart sound that will begin soon. Consequently, the chance of missing listening to a heart sound will be reduced. The whole listening process of heart sounds will be more accurate.

In addition to the notification function, the stethoscope device provided by the present invention also possesses the ability to measure heart sound signals and electrocardiogram signals at the same time. Moreover, with a storage unit, the stethoscope device provided by the present invention can store the original heart sound signals and electrocardiogram signals to the storage unit. The storage unit can then output these signals to other units that can display signals.

Although the present invention has been explained in relation to its preferred embodiment, it is to be understood that many other possible modifications and variations can be made without departing from the spirit and scope of the invention as hereinafter claimed.

What is claimed is:

1. A stethoscope device, comprising:
a heart sound sensor comprising a sensing portion to detect a heart sound signal having a periodic first heart sound and a periodic second heart sound;
an electrocardiogram sensor comprising two electrodes to detect an electrocardiogram signal having a periodic R wave;
a microprocessor for extracting amplitudes of the R wave from the electrocardiogram signal and analyzing appearing positions of the R wave to provide appearing positions of a periodic notification pulse wave to a predetermined reference signal and combine the reference signal with the heart sound signal to form a sound signal; and
an output unit to output the sound signal;
wherein each pulse of the notification pulse wave of the sound signal provides a notification sound.

2. The stethoscope device as claimed in claim 1, wherein the notification pulse wave of the reference signal delays for a predetermined time compared to the corresponding R wave.

3. The stethoscope device as claimed in claim 2, wherein the predetermined time is in the range of 0.06 seconds to 0.07 seconds.

4. The stethoscope device as claimed in claim 3, wherein the predetermined time is 0.065 seconds.

5. The stethoscope device as claimed in claim 2, wherein the notification sound precedes the first heart sound.

6. The stethoscope device as claimed in claim 2, further comprising an LED emitting light together with the notification sound.

7. The stethoscope device as claimed in claim 1, further comprising a storage unit to store the sound signal.

8. The stethoscope device as claimed in claim 7, wherein the storage unit is a SD card.

9. The stethoscope device as claimed in claim 1, wherein the output unit is a headphone.

10. The stethoscope device as claimed in claim 1, further comprising a first amplifier connecting to the heart sound sensor and the electrocardiogram sensor to amplify the heart sound signal and the electrocardiogram signal.

11. The stethoscope device as claimed in claim 10, further comprising an analog-to-digital converter to convert the amplified heart sound signal and the amplified electrocardiogram signal from analog signals to digital signals to be inputted to the microprocessor.

12. The stethoscope device as claimed in claim 1, further comprising a second amplifier connecting in between the microprocessor and the output unit to amplify the sound signal to be outputted by the output unit.
